(19) United States
(12) Patent Application Publication
(10) Pub. No.: US 2007/0020710 A1
(43) Pub. Date: Jan. 25, 2007

(54) Antibodies that spefically recognize inactive PSA, and uses thereof

(75) Inventors: Jean-Philippe Charrier, Ecully (FR); Colette M. Jolivet-Reynaud, Sain Bonnet de Mure (FR); Sandrine Michel, Lyon (FR)

Correspondence Address: OLIFF & BERRIDGE, PLC, P.O. BOX 19928, ALEXANDRIA, VA, 22320, US

(73) Assignee: BIO MERIEUX, Marcy-L'Etoile (FR)

(21) Appl. No.: 11/525,990

(22) Filed: Sep. 25, 2006

Related U.S. Application Data
(62) Division of application No. 10/257,029, filed on Feb. 6, 2003.

(30) Foreign Application Priority Data
Apr. 10, 2000 (FR) 00 04591

(51) Int. Cl. G01N 33/574

(52) U.S. Cl. 435/7.23

(57) ABSTRACT

An immunoassay for the quantitative determination of inactive, non-zymogen, free PSA in a biological sample taken from an individual, including contacting the biological sample with a reagent, where the reagent comprises an antibody or antibody fragments that bind specifically to inactive, non-zymogen, free PSA, the antibody being a purified polyclonal antibody or a monoclonal antibody, and evaluating the amount of inactive, non-zymogen, free PSA bound to the antibodies or antibody fragments.

This application is a divisional application of U.S. patent application Ser. No. 10/257,029 filed Feb. 6, 2003, which in turn is a National Stage Application of International Patent Application No. PCT/FR01/01094 filed Apr. 10, 2001,

### BACKGROUND

PSA is produced by the glandular epithelium of the human prostate, probably in an inactive zymogen form (Lundwall et al., FEBS Lett 1987), and is secreted into the seminal fluid in its active form (Lilja, J. Clin. Invest. 1985). The biological activity of PSA in the seminal fluid is linked to its limited proteolytic fragmentation of the predominant proteins secreted by seminal vesicles (Lilja, J. Clin. Invest. 1985; Lilja et al., J. Clin. Invest. 1987; McGee et al., Biol. reprod. 1988).

PSA is the main marker for prostate cancer, which will affect, during his life, one man in six in Western countries. This protease of the kallikrein family, mainly secreted by the prostatic epithelium, is found at a concentration of 0.5 to 5 mg/ml in seminal fluid and at a concentration a million times lower in a patient's serum. Thus, PSA is normally found at a concentration of less than 2.5 ng/ml in the serum. However, this concentration increases in principle, notably in the event of a cancer of the prostate and moderately in the event of benign modifications such as benign prostatic hypertrophy (BPH) or acute prostatitis.

The protein sequence of PSA has been determined. It is a glycoprotein comprising 237 amino acids (“Molecular cloning of human prostate specific antigen cDNA”, Lundwall A., Lilja H., 1987, FEBS Lett 214: 317-322).

A diagnostic method has been proposed, consisting in measuring the serum PSA concentration and then comparing it with a threshold value of 4 ng/ml. However, it has been noted that this diagnostic method leads to three patients out of four being wrongly suspected, which is prejudicial. In addition, this method does not make it possible to diagnose 30 to 45% of cases of cancers confined to the gland, which nevertheless constitutes a potentially curable, early stage of the disease, the diagnosis of which would therefore be particularly desirable. The relatively unsatisfactory nature of this method is, moreover, demonstrated by the study reported in the article “Prostate Cancer Detection in Men With Serum PSA Concentrations of 2.6 to 4.0 ng/ml and Benign Prostate Examination”, Catalona et al., JAMA, 14 May 1997—Vol. 277, No. 18, which shows that the threshold value should be less than 4 ng/ml for early screening of prostate cancer.

Moreover, it has recently been shown that, in serum, PSA associates with protease inhibitors, such as a 1 antichymotrypsin (ACT) and a 2 macroglobulin (A2M). These associations lead to inactivation of the chymotrypsin activity of PSA, as was demonstrated in the article “Enzymatic activity of prostate specific antigen and its reactions with ultracellular serine proteinase inhibitors”, A. Christensson, C. B. Laurell, H. Lilja, 1990 Eur. J. Biochem 194: 755-763. The use of the free PSA to total PSA ratio was then proposed in order to improve the specificity of diagnosis.

Thus, patent application WO A 97/12245 describes a method for diagnosing an adenocarcinoma of the prostate without biopsy. This method consists in measuring, in the serum or blood of patients, the total amount of PSA. If this value is between 2.5 and 20 ng/ml, the concentration of free PSA is also measured. The free PSA to total PSA ratio is then calculated. If this ratio is less than 7%, the diagnosis leans toward an adenocarcinoma of the prostate.

However, the use of a threshold at 7% for diagnosing a cancer of the prostate is disputed by many authors, as shown in the publication by Lein et al. “Relation of free PSA/total PSA in serum for differentiating between patients with prostatic cancer and benign prostate hyperplasia: which cutoff should be used?” In that document, published in the review Cancer Investigation, 16(1), 45-49, 1998, it was demonstrated that it is difficult, via this ratio, to systematically differentiate a cancer of the prostate from a BPH.

For these reasons, the applicant, in a patent application WO A 00/02052, filed under the priority of Jul. 3, 1998, was interested in the presence in the serum of a cleaved form of PSA. The molecular forms of serum PSA of patients suffering from cancer or from BPH had been mapped by two-dimensional electrophoresis, combined with detection by chemiluminescence, in order to observe all of the forms of PSA, i.e. the free, complexed and cleaved forms.

The electrophoretic profiles of sera from patients suffering from adenocarcinoma of the prostate are relatively homogeneous, exhibiting essentially the noncleaved form of PSA, whereas those from individuals suffering from BPH may comprise a relatively large proportion of cleaved form and slightly more basic spots without cleaved form. The increase in the free serum PSA to total serum PSA ratio observed in patients suffering from BPH is therefore thought to be essentially linked to the existence of cleaved PSA, which might be enzymatically inactive and therefore incapable of binding to ACT, and also to the presence of a free form of PSA slightly more basic than the active free form of PSA, which might correspond to the inactive, zymogen form of PSA.

Based on these observations, the applicant has described methods for diagnosing adenocarcinoma of the prostate, comprising the quantification, after separation by two-dimensional electrophoresis, of cleaved and/or noncleaved free PSA and the use of these values in order to establish a diagnosis.

However, these methods require two-dimensional electrophoresis to be performed. They are, consequently, quite expensive and require considerable handling time.

### SUMMARY

The present invention relates to antibodies or antibody fragments which bind specifically to prostate-specific antigen (PSA) in its inactive free form, and also to the hybridomas that produce such antibodies.

The present invention also relates to reagents for immunoassays containing such antibodies and also to immunoassays comprising the use of such reagents.

The present invention also relates to a method for diagnosing an adenocarcinoma of the prostate in an individual suspected of suffering from such a pathology.

The present invention also relates to a diagnostic method which makes it possible to differentiate between a benign pathology of the prostate and an adenocarcinoma of the prostate in an individual suspected of suffering from one of these diseases.

Finally, the present invention relates to the diagnostic kit for diagnosing an adenocarcinoma of the prostate in an individual suspected of suffering from an adenocarcinoma or for differentiating between a benign pathology of the prostate and an adenocarcinoma of the prostate in an individual suspected of suffering from one of these diseases.

### DETAILED DESCRIPTION OF EMBODIMENTS

It has now been discovered that it is possible to have methods for diagnosing adenocarcinomas of the prostate which do not have the abovementioned drawbacks and which have excellent specificity and sensitivity, by using immunoreagents. In has in fact been discovered that it is possible to obtain antibodies directed specifically against inactive free PSA. It has also been discovered that such antibodies may advantageously be used in particular in methods for diagnosing cancer of the prostate and BPH.

A subject of the present invention is thus antibodies or antibody fragments which bind specifically to inactive free PSA, said antibodies being purified polyclonal antibodies or monoclonal antibodies.

In the present application, the term “antibody fragment” is generally intended to mean any antibody fragment having conserved the specificity of the antibody of origin, and in particular fragments of the Fab and F(ab1)2 type. In the present application, the word “antibody” also subsequently denotes antibody fragments when appropriate.

The expression “antibodies which bind specifically to a given form of PSA” is intended to denote, of course, an antibody which binds essentially to said form of PSA. For example, the product which is bound to the antibody consists of at least 80%, and preferably of at least 90%, of said form of PSA.

In the present application, the expression “inactive free PSA” is intended to mean a free form of PSA, i.e. a noncomplexed form, in other words a form not associated with its inhibitors such as ACT. In addition, this free form is inactive. The term “inactive” denotes a form of PSA that exhibits a proteolytic activity (in particular of the chymiotrypsin type) clearly lower than that of the free PSA present in the serum of healthy individuals, for example lower by at least 80% or more.

A subject of the present invention is in particular monoclonal antibodies or antibody fragments which bind specifically to inactive free PSA.

A subject of the present invention is also the hybridomas that produce monoclonal antibodies as defined above.

The production of hybridomas is well known and will not be reiterated here. The hybridomas that recognize specifically inactive free PSA are selected, as indicated below. It was discovered that certain clones produced antibodies which, when attached to a chromatography column, retained only a fraction of the total serum PSA of various individuals, and that this fraction was, with some of these antibodies, a free and substantially inactive form (as defined above) of PSA.

Moreover, it was noted that the PSA fractions not retained by these antibodies still contained free PSA and had conserved practically all the proteolytic activity of the starting serum.

It was thus possible to obtain fractions enriched in inactive free PSA, which were then used to systematically test hybridoma clones obtained as previously.

Among the hybridomas obtained after immunization of mice with PSA of seminal origin marketed by the company SCIPAC, it was noted that approximately 1.5% produced antibodies that bind specifically to the inactive free PSA thus obtained.

A subject of the present invention is also a reagent for immunoassay containing antibodies or antibody fragments as defined above.

In the reagents according to the invention, the antibodies or antibody fragments which bind specifically to inactive free PSA can be used as such or else in particular in a form attached to a solid support and/or linked to a label.

The attachment of antibodies or of antibody fragments to a solid support is well known. The support may be made with any solid, biological or synthetic material with adsorbent properties or capable of attaching a coupling agent. Materials are known and described in the literature. Among the solid materials capable of attaching the antibodies by adsorption, mention should be made, for example, of polystyrene, polypropylene, latex, etc. Among the materials that make it possible to attach the antibodies covalently using a coupling agent, mention may in particular be made of dextran, cellulose, etc. The support may, for example, be in the form of disks, of tubes, of beads or plates, in particular of microtitration plates.

Linking an antibody or an antibody fragment to a label, whether it is a radioactive, enzymatic or colored label or any other type of label commonly used in immunological techniques, is well known and described in the literature.

A subject of the present invention is also an immunoassay for the quantitative determination of inactive free PSA in a biological sample taken from an individual, in which said sample is brought into contact with a reagent as defined above, and the amount of inactive free PSA bound to the antibodies of the reagent is evaluated.

The biological sample taken from the individual is generally a blood or serum sample. If desired, it may, of course, have undergone a concentration or dilution step prior to it being brought into contact with the reagent containing antibodies or antibody fragments which bind specifically to inactive free PSA.

The quantification of the inactive free PSA bound to the antibodies or antibody fragments may be carried out in various well-known ways.

For example, the amount of inactive free PSA may be evaluated using sandwich-type assays. According to a particular embodiment, a reagent for immunoassay, in which the antibodies or antibody fragments which bind specifically to inactive free PSA are attached to the support, is brought into contact with the biological sample for which it is desired to determine the inactive free PSA content, and then, after washing, said support is brought into contact with a second labeled antibody which binds to PSA. After further washing, the amount of label attached is measured and the inactive free PSA content is deduced by comparison with a standard curve established beforehand.

According to another embodiment of the immunoassays of the invention, the amount of a form of PSA other than the inactive free form, present in a sample of the same nature taken from the same individual, is also evaluated.

The expression “sample of the same nature taken from the same individual” is intended to mean either two fractions from the same sampling, or two samples derived from two different samplings but which must be of the same nature, for example serum samples.

Of course, when the amounts measured are intended to be compared, it is advisable for these values to be comparable. In other words, the values measured should be corrected to the same dilution or possible concentration of the sample and also to the same volume.

The forms other than the inactive free form of PSA are in particular complexed PSA, total free PSA, active free PSA and total PSA, i.e. all the active or inactive, free or complexed forms of PSA.

The assaying of each of these various forms, in particular using specific antibodies, is known.

Complexed PSA is assayed, for example, using antibodies described in patent application WO A 98/22509.

Total PSA is assayed, for example, using antibodies described by H. Nagasaki et al. (1999), Clin. Chem. 45: 4486-496.

Free PSA is assayed, for example, using antibodies described in patent application WO 92/01936, or using monoclonal antibodies marketed by CHUGAI (Japan).

A subject of the present invention is in particular an immunoassay as defined above, in which active free PSA is also assayed. The amount of active free PSA can be evaluated in various ways.

Thus, according to a first embodiment, the amount of active free PSA is evaluated using antibodies which bind specifically to active free PSA, which can be obtained by immunization with fractions of total PSA or with fractions enriched in active free PSA. The amount of active free PSA can also be evaluated indirectly by evaluating the amount of total free PSA, from which the amount of inactive free PSA already evaluated as above is subtracted.

A subject of the present invention is also an immunoassay for determining:
the ratio of the amount of inactive free PSA to the amount of total free PSA, or
the ratio of the amount of inactive free PSA to the amount of active free PSA, or
the ratio of the amount of active free PSA to the amount of total free PSA, or
the ratio of the amount of inactive free PSA to the amount of total PSA, or
the ratio of the amount of inactive free PSA to the amount of complexed PSA, or
the ratio of the amount of active free PSA to the amount of total PSA, or
the ratio of the amount of active free PSA to the amount of complexed PSA, or
   the inverses of these ratios, or combinations of these ratios, in a biological sample taken from an individual, in which:
the amount of inactive free PSA in a biological sample taken from an individual is evaluated by bringing said sample into contact with a reagent according to the invention as defined above,
one of the amounts chosen from the amount of active free PSA, the amount of total free PSA, the amount of total PSA and the amount of complexed PSA is evaluated on a sample of the same nature taken from the same individual, and
said ratio or said inverse ratio, or said combination of ratios, is determined.

The present invention relates to various diagnostic methods which, in general, apply to individuals having a serum PSA concentration of greater than 2 ng/ml or greater than 2.5 ng/ml.

A subject of the present invention is also a method for diagnosing an adenocarcinoma of the prostate in an individual suspected of suffering from said adenocarcinoma, without performing a biopsy, in which one or more immunoassays as defined above are carried out.

It has in fact been discovered that the concentration (for example serum concentration) of inactive free PSA is clearly higher in the case of a BPH than in that of an adenocarcinoma of the prostate.

The invention therefore relates in particular to a method for diagnosing BPH or for differentiating between a cancer of the prostate or a BPH, in which the inactive free PSA content in a sample originating from an individual is measured, and this content is compared with a scale of predetermined values, said values being those observed in patients suffering from a recognized BPH and those observed in patients suffering from a recognized adenocarcinoma of the prostate, and it is concluded, from the result of this comparison, either that there is a BPH or that there is an adenocarcinoma of the prostate. This method, in practice, amounts to the same thing as comparing the value measured to a threshold value.

Of course, these variations in the inactive free PSA content influence various ratios, such as ratios of the amount of inactive free PSA to the amount of total free PSA or to the amount of total PSA. It is also possible to determine other ratios, as will be specified below.

It is known that, in general, the results of immunoassays depend to a large extent on the characteristics of specificity and of affinity of the antibodies used, and that these characteristics influence the values measured with these antibodies. It is therefore understood that it is not possible to give precise threshold values and that threshold values suited to each antibody used can be determined in each case by simple routine experiments.

It should be clearly understood that the term “threshold value” herein is either a discrete value or a range of values corresponding to a region of uncertainty. Of course, when the value measured is included in the range of uncertainty, those very close to the threshold value in the case of a discrete value, it is not possible to draw a definitive conclusion and further investigations should be carried out.

Of course, when a threshold value has been determined for a given type of ratio, it is possible to deduce threshold values corresponding to other types of ratio.

A subject of the present invention is in particular a diagnostic method as defined above, in which, in addition, the value of the ratio thus determined is compared with a predetermined threshold value chosen, depending on the type of ratio used, such that the values of said ratio higher or, respectively, lower, than said threshold value are representative of the values observed in patients suffering from a recognized adenocarcinoma of the prostate, and such that, conversely, the values of said ratio lower or, respectively, higher, than said threshold value are representative of the values observed in individuals recognized not to be suffering from an adenocarcinoma of the prostate, and it is concluded, from the result of said comparison, either that an adenocarcinoma of the prostate is present, or that one is not present.

A subject of the present invention is also a diagnostic method which makes it possible to differentiate between a benign pathology of the prostate and an adenocarcinoma of the prostate in an individual suspected of suffering from one of these diseases, in which an immunoassay as defined above is carried out.

A subject of the present invention is in particular such a method in which, in addition, the value of one of the ratios mentioned above is compared with a predetermined differentiation value chosen, depending on the type of ratio used, such that the values of said ratio which are higher or, respectively, lower, than said differentiation value are representative of the values observed in patients suffering from a recognized benign pathology of the prostate, and such that, conversely, the values of said ratio which are lower or, respectively, higher, than said differentiation value are representative of the values observed in individuals recognized to be suffering from a recognized adenocarcinoma of the prostate, and it is concluded, from the result of said comparison, that there is either a benign pathology of the prostate or an adenocarcinoma of the prostate.

In practice, the differentiation values are analogous to the threshold values discussed above.

A subject of the present invention is also a method for diagnosing an adenocarcinoma of the prostate in an individual suspected of suffering from said adenocarcinoma, in which:
the amount of active free PSA and/or the amount of inactive free PSA in a sample taken from this individual is determined,
where appropriate, the amount of a form of PSA other than active free PSA and other than inactive free PSA, in a sample of the same nature taken from the same individual, is determined,
the value of one of the following ratios is determined:
   ratio of the amount of inactive free PSA to the amount of active free PSA,
   ratio of the amount of inactive free PSA to the amount of total free PSA,
   ratio of the amount of inactive free PSA to the amount of total PSA,
   ratio of the amount of inactive free PSA to the amount of complexed PSA,
   ratio of the amount of active free PSA to the amount of total free PSA,
   ratio of the amount of active free PSA to the amount of total PSA,
   ratio of the amount of active free PSA to the amount of complexed PSA, or
   the inverses of these ratios, or combinations of these ratios
the value of the ratio thus determined is compared with a predetermined threshold value chosen, depending on the type of ratio used, such that the values of said ratio higher or, respectively, lower, than said threshold value are representative of the values observed in patients suffering from a recognized adenocarcinoma of the prostate, and such that, conversely, the values of said ratio lower or, respectively, higher, than said threshold value are representative of the values observed in individuals recognized not to be suffering from an adenocarcinoma of the prostate, and it is concluded, from the result of said comparison, either that an adenocarcinoma of the prostate is present, or that one is not present.

A subject of the present invention is also a diagnostic method which makes it possible to differentiate between a benign pathology of the prostate and an adenocarcinoma of the prostate in an individual suspected of suffering from one of these diseases, in which:
the amount of active free PSA and/or the amount of inactive free PSA in a sample taken from this individual is determined,
where appropriate, the amount of a form of PSA other than active free PSA and other than inactive free PSA, in a sample of the same nature taken from the same individual, is determined,
the value of one of the following ratios is determined:
   ratio of the amount of inactive free PSA to the amount of active free PSA,
   ratio of the amount of inactive free PSA to the amount of total free PSA,
   ratio of the amount of inactive free PSA to the amount of total PSA,
   ratio of the amount of inactive free PSA to the amount of complexed PSA,
   ratio of the amount of active free PSA to the amount of total free PSA,
   ratio of the amount of active free PSA to the amount of total PSA,
   ratio of the amount of active free PSA to the amount of complexed PSA, or
   the inverses of these ratios, or combinations of these ratios,
the value of the ratio thus determined is compared with a predetermined differentiation value chosen, depending on the type of ratio used, such that the values of said ratio which are higher or, respectively, lower, than said differentiation value are representative of the values observed in patients suffering from a recognized benign pathology of the prostate, and such that, conversely, the values of said ratio which are lower or, respectively, higher, than said differentiation value are representative of the values observed in individuals recognized as suffering from a recognized adenocarcinoma of the prostate, and it is concluded, from the result of said comparison, either that there is a benign pathology of the prostate or that there is an adenocarcinoma of the prostate.

A subject of the present invention is also a diagnostic kit for diagnosing an adenocarcinoma of the prostate in an individual suspected of suffering from said adenocarcinoma, or for differentiating between a benign pathology of the prostate and an adenocarcinoma of the prostate in an individual suspected of suffering from one of these diseases, said kit comprising:
means for assaying inactive free PSA in a biological sample,
means for assaying active free PSA in a biological sample of the same nature, and, optionally,
means for assaying a form of PSA other than inactive free PSA and other than active free PSA, in a biological sample of the same nature.

Among the means that can be used in said kit, antibodies that bind specifically to the form of PSA, the quantification of which is desired, are in particular used.

The following examples illustrate the invention without, however, limiting it.

### EXAMPLES

### Example 1

Production and Characterization of Monoclonal Antibodies

Production of Monoclonal Antibodies that Bind to PSA

4- to 6-week-old female BALB/c JYCo mice (IFFA Credo) are immunized by intraperitoneal injection with 15 mg of purified PSA (PSA of seminal origin provided by SCIPAC) emulsified with an equal volume of Freund's complete adjuvant followed by five injections of incomplete adjuvant, the injections being two weeks apart. Four days after the final injection, the cells of the spleen are taken and fused, according to the method of Kohler and Milstein, with cells of the mouse myeloma cell line Sp 2/0-Ag14. The cells are then cultured for 12 to 14 days. The culture supernatants are recovered and are then subjected to an assay according to an ELISA method, in which the solid phase is coated with the antigen used for immunization. The positive colonies, i.e., the colonies reacting positively with respect to PSA, are subcloned twice by limiting dilution. Selected clones are cultured by ascites in a known manner. The IgG fraction of each ascites fluid is purified by chromatography on a protein A-Sepharose 4 FF column, according to the manufacturer's instructions (PHARMACIA).

### Example 2

Determination of the Specificity of the Monoclonal Antibodies with Respect to the Free, Complex and Total Forms of PSA

96-well plates (Nunc) are coated with 100 ml of a solution comprising 1 mg/l of noncomplexed PSA (supplied by SCIPAC), of PSA-ACT complex (supplied by SCRIPPS Laboratories) or of ACT (also supplied by SCRIPPS Laboratories) in 0.05 mol/l of carbonate buffer, pH 9.6. After overnight incubation at ambient temperature, the plates are washed three times with a phosphate buffered saline solution (PBS; 50 mmol/l phosphate buffer, pH 7.2, 150 mmol/l NaCl) containing 0.5 ml/l of Tween20 (PBS-T), and blocked for 1 hour at 37° C. with PBS containing 10 g/l of dried milk extract. The plates are then washed a second time with PBS-T. 100 ml of ascites fluid diluted to from 10-1 to 10-6 in PBS-T are added to each well of each plate, which are then incubated for one hour at 37° C. The plates are then washed with PBS-T, and 100 ml of anti-mouse goat IgG conjugate to alkaline phosphatase (Jackson ImmunoResearch Laboratories) at a dilution of 1:2 000 in PBT-T containing 10 g/l of bovine serum albumin (BSA) (SIGMA) are added to each well of each plate. The plates are incubated for one hour at 37° C. and are then washed with PBS-T.

The substrate, a solution of p-nitrophenylphosphate (SIGMA), is added for 30 minutes. The enzymatic activity is blocked with a 1 mol/l NaOH solution and the absorbency is measured at 405 nm with an ELISA plate reader (BioMerieux).

It was thus possible to select several antibodies that recognize free PSA but which do not recognize either complex PSA or ACT.

### Example 3

Characterization of the Specificity with Respect to the Inactive and Active Forms of PSA

In order to continue the characterization of the specificity of the antibodies, PSA having the desired characteristics was prepared.

Thus, the LNCaP cell line was made to synthesize PSA, according to the technique described in the articles “LNCaP Produces both putative zymogen and inactive, free form of prostate specific antigen”, E. Corey et al., 1998, Prostate 35:135-143, “Androgen-sensitive human prostate cancer cell, LNCaP, produces both N-terminally mature and truncated prostate specific antigen isoform”, A. Herrala et al., 1997, Eur. J. Biochem. 255:329-335, and “Production of milligram concentration of free prostate specific antigen (fPSA) from LNCaP cell culture: Difference between fPSA from LNCaP cell and seminal plasma.”, J. T. Wu et al., 1998, J. Clin. Lab. Anal. 12:6-13.

The PSA thus produced by the LNCaP cell line is composed of zymogen PSA, of active enzymatically mature PSA, of inactive whole PSA and also of inactive, cleaved or truncated forms of PSA.

In addition, the line is cultured in the presence of fetal calf serum, which contains ACT, a proportion of PSA-ACT complex thus also being present. This PSA of cellular origin is therefore a good tool for demonstrating the specificity of recognition of the various anti-free PSA monoclonal antibodies obtained in example 2.

LNCaP cells are cultured in a synthetic medium to which fetal calf serum has been added. The various culture supernatants containing the secreted PSA are pooled and divided into identical fractions of 380 ml.

Immunoabsorbents are prepared by coupling, in an identical manner, the anti-PSA monoclonal antibodies obtained in example 2 to sepharose resin. Affinity columns containing identical amounts of resin are thus formed. Each fraction of cellular PSA is immunopurified on one of these affinity columns, and the PSA bound by each monoclonal antibody is eluted from the column, first with an acid solution (0.1 M glycine, pH 2.8) and then with a basic solution (0.1 M DEA, pH 11). The eluates are then immediately neutralized so as to preserve the activity of the PSA.

The PSA content in the various fractions obtained is analyzed: after having been quantified, the enzymatic activity is measured. The various forms of PSA contained in the eluates are then characterized by Western Blotting after SDS-PAGE.

(i) Amount of PSA Contained in the Eluates

The amount of PSA in each acid or basic fraction is determined by an ELISA method using an anti-PSA polyclonal antibody in order to be sure that the PSA will be assayed in its entirety.

Table 1 below gives the results obtained with three particular antibodies, specific for the free form of PSA, obtained in example 2.

TABLE 1
Assaying of PSA immunopurified with the anti-PSA monoclonal antibodies
(“acid elution” and “basic elution) fractions”)

| | ACID ELUTION | | | BASIC ELUTION | | |
|---|---|---|---|---|---|---|
| | | Fraction | Amount of | | Fraction | Amount of |
| Monoclonal | Concentration | volume | PSA eluted | Concentration | volume | PSA eluted |
| antibodies | (μg/ml) | (ml) | (μg) | (μg/ml) | (ml) | (μg) |
| 1 | 0.289 | 3.6 | 1.0 | 0.003 | 9.5 | 0.0 |
| 2 | 0.412 | 7.1 | 2.9 | 0.001 | 10.0 | 0.0 |
| 3 | 0.593 | 5.1 | 3.0 | 0.006 | 9.4 | 0.1 |
| Starting cell | 0.061 | 380.0 | 23.2 | | | |
| supernatant | | | | | | |

The results of table 1 show that the PSA is found exclusively in the “acid eluate” fractions, and that not all the monoclonal antibodies have retained the same amount of PSA.

(ii) Specific Enzymatic Activity of the PSA Purified using these Antibodies

The enzymatic activity of the PSA contained in the “acid elution” fractions is measured on a substrate of the chymotrypsin type, S-2586 (MeO-Suc-Arg-Pro-Tyr-pNA.HCl), obtained from Chromogenix AB (Molndal, Sweden). This activity is determined on 60 ml of sample, and the specific activity is calculated from the concentration determined in the preceding paragraph.

The results are given in table 2 below.

TABLE 2

| | Specific activity of the PSA |
|---|---|
| Monoclonal antibodies | (Δ OD/min/μg of PSA × 1 000) |
| 1 | 21.6 |
| 2 | 1.2 |
| 3 | 0.9 |
| Starting cell supernatant | 11.6 |

The results show that the specific enzymatic activity of the acid eluate obtained with the anti-PSA antibody No. 1 is twice as high as the activity of the starting cell supernatant, before immunopurification.

This antibody therefore enriched the purified PSA in enzymatically active form. In addition, this antibody No. 1 recognizes a free form of PSA. Consequently, the antibody No. 1. recognizes essentially the active free form of PSA.

The specific enzymatic activity of the acid eluates obtained with the antibodies Nos. 2 and 3 is very low. This shows that these antibodies essentially retained PSA in its inactive form. Since these antibodies are specific for a free form of PSA, they recognize essentially PSA in its inactive free form.

In addition, it is known that trypsin is capable of transforming zymogen PSA into enzymatically active PSA. For this reason, in order to determine whether zymogen PSA had been retained, during the affinity chromatography operations described above, by the anti-PSA monoclonal antibodies, the enzymatic activity of the “acid eluates” was measured in the presence of trypsin. The specific activity of the eluates, determined with or without trypsin, is identical. Consequently, the acid eluates obtained with the antibodies No. 1, 2, and 3, as described above, do not contain any PSA in zymogen form.

Therefore, none of the antibodies 1, 2, or 3 recognizes the zymogen form of PSA.

Immunoanalysis Carried Out After SDS-PAGE

The various forms of PSA present in the eluates derived from the columns to which the antibodies No. 1, 2, and 3 are attached were separated by acrylamide gel electrophoresis under reducing conditions, and detection of the PSA by Western blotting is carried out with a monoclonal antibody which recognizes total PSA.

The Western blotting carried out confirmed that PSA is present in all the samples tested originating from the acid elution, in proportions in correlation with the amounts determined in table 1, whereas the samples originating from the basic elutions do not contain any.

The Western blotting carried out also confirms that the antibodies No. 1, 2, and 3 did not retain the PSA-ACT complex. Finally, by comparison with the PSA obtained as indicated in example 3, used as a control, the acid elutions contain PSA forms that have a normal molecular weight (33 kDa), and also forms that have a lower molecular weight.

What is claimed is:

1. An immunoassay for the quantitative determination of inactive, non-zymogen, free PSA in a biological sample taken from an individual, comprising:
contacting the biological sample with a reagent, wherein the reagent comprises an antibody or antibody fragments that bind specifically to inactive, non-zymogen, free PSA, the antibody being a purified polyclonal antibody or a monoclonal antibody; and
evaluating the amount of inactive, non-zymogen, free PSA bound to the antibodies or antibody fragments.

2. The immunoassay according to claim 1, further comprising:
evaluating an amount of a form of PSA other than the inactive, non-zymogen, free form, present in a biological sample of the same nature taken from the same individual.

3. The immunoassay according to claim 2, in which the other form of PSA is chosen from the group consisting of active free PSA, total free PSA, complexed PSA, and total PSA.

4. The immunoassay according to claim 3, in which the other form is active free PSA.

5. An immunoassay for determining a ratio of amounts of different forms of PSA in a biological sample taken from an individual, comprising:
evaluating an amount of inactive, non-zymogen, free PSA according to claim 1;
evaluating an amount of at least one form of PSA, other than the inactive, non-zymogen, free form, present in a biological sample of the same nature taken from the same individual, wherein the group of different forms of PSA consists of:
active free PSA,
complexed PSA,
total free PSA, and
total PSA; and
determining a value of a ratio, inverse ratio, or any combination of ratios from the group of ratios consisting of:
a ratio of the amount of inactive, non-zymogen, free PSA to the amount of total free PSA,
a ratio of the amount of active free PSA to the amount of total free PSA,
a ratio of the amount of inactive, non-zymogen, free PSA to the amount of total PSA,
a ratio of the amount of inactive, non-zymogen, free PSA to the amount of complexed PSA,
a ratio of the amount of active free PSA to the amount of total PSA, and
a ratio of the amount of active free PSA to the amount of complexed PSA.

6. A method for diagnosing a benign pathology of the prostate or for differentiating between a benign pathology of the prostate and an adenocarcinoma of the prostate in an individual, comprising measuring an amount of inactive, non-zymogen, free PSA content in a sample originating from the individual.

7. The method according to claim 6, further comprising:
quantitatively determining by an immunoassay amounts of inactive, non-zymogen, free PSA in a biological sample taken from an individual, comprising contacting the biological sample with a reagent, wherein the reagent comprises an antibody or antibody fragments that bind specifically to inactive, non-zymogen, free PSA, the antibody being a purified polyclonal antibody or a monoclonal antibody.

8. The method according to claim 6, further comprising:
comparing an amount of inactive, non-zymogen, free PSA content with a scale of predetermined values, the values being those observed in patients suffering from a recognized benign pathology of the prostate and those observed in patients suffering from a recognized adenocarcinoma of the prostate; and
concluding from the result of the comparison whether there is a benign pathology of the prostate or an adenocarcinoma of the prostate.

9. A method for diagnosing an adenocarcinoma of the prostate in an individual suspected of suffering from said adenocarcinoma without performing a biopsy, wherein the immunoassay according to claim 5 is carried out.

10. The method according to claim 9, further comprising:
comparing the value of the ratio thus determined with a first predetermined threshold value chosen corresponding to the type of ratio used, wherein the first threshold value is representative of the values observed in patients suffering from a recognized adenocarcinoma of the prostate;
comparing the value of the ratio thus determined with a second predetermined threshold value chosen corresponding to the type of ratio used, wherein the second threshold value is representative of the values observed in individuals recognized not to be suffering from an adenocarcinoma of the prostate; and
concluding from the results of the comparisons whether there is present an adenocarcinoma of the prostate.

11. A diagnostic method to differentiate between a benign pathology of the prostate and an adenocarcinoma of the prostate in an individual suspected of suffering from one of these diseases, comprising conducting the immunoassay according to claim 5.

12. The method according to claim 11, further comprising:
comparing the value of the ratio thus determined with a first predetermined differentiation value chosen corresponding to the type of ratio used, wherein the first differentiation value is representative of the values observed in patients suffering from a recognized benign pathology of the prostate;
comparing the value of the ratio thus determined with a second predetermined differentiation value chosen corresponding to the type of ratio used, wherein the second differentiation value is representative of the values observed in individuals recognized to be suffering from an adenocarcinoma of the prostate; and
concluding from the results of the comparisons whether there is present a benign pathology of the prostate or an adenocarcinoma of the prostate.

13. A method for diagnosing an adenocarcinoma of the prostate in an individual suspected of suffering from said adenocarcinoma, comprising:
(i) evaluating an amount of at least one of active free PSA and inactive, non-zymogen, free PSA in a biological sample taken from the individual;
(ii) evaluating an amount of at least one other form of PSA, different from the form selected in step (i), present in a biological sample of the same nature taken from the same individual, wherein the group of different forms of PSA consists of:
inactive, non-zymogen, free PSA,
active free PSA,
complexed PSA,
total free PSA, and
total PSA;
(iii) determining a value of a ratio, inverse ratio, or any combination of ratios from the group of ratios consisting of:
a ratio of the amount of inactive, non-zymogen, free PSA to the amount of total free PSA,
a ratio of the amount of active free PSA to the amount of total free PSA,
a ratio of the amount of inactive, non-zymogen, free PSA to the amount of total PSA,
a ratio of the amount of inactive, non-zymogen, free PSA to the amount of complexed PSA, and
a ratio of the amount of active free PSA to the amount of total PSA;
(iv) comparing the value of the ratio thus determined with a first predetermined threshold value chosen corresponding to the type of ratio used, wherein the first threshold value is representative of the values observed in patients suffering from a recognized adenocarcinoma of the prostate;
(v) comparing the value of the ratio thus determined with a second predetermined threshold value chosen corresponding to the type of ratio used, wherein the second threshold value is representative of the values observed in individuals recognized not to be suffering from an adenocarcinoma of the prostate; and
(vi) concluding from the results of the comparisons from steps (iv) and (v) whether there is present an adenocarcinoma of the prostate.

14. A diagnostic method to differentiate between a benign pathology of the prostate and an adenocarcinoma of the prostate in an individual suspected of suffering from one of these diseases, comprising:
(i) determining an amount of at least one of active free PSA and inactive, non-zymogen, free PSA in a biological sample taken from the individual;
(ii) determining an amount of at least one other form of PSA, different from the form selected in step (i), present in a biological sample of the same nature taken from the same individual, wherein the group of different forms of PSA comprises:
inactive, non-zymogen, free PSA,
active free PSA,
complexed PSA,
total free PSA, and
total PSA;
(iii) determining a value of a ratio, inverse ratio, or any combination of ratios from the group of ratios consisting of:
a ratio of the amount of inactive, non-zymogen, free PSA to the amount of total free PSA, a ratio of the amount of active free PSA to the amount of total free PSA,
a ratio of the amount of inactive, non-zymogen, free PSA to the amount of total PSA,
a ratio of the amount of inactive, non-zymogen, free PSA to the amount of complexed PSA,
a ratio of the amount of active free PSA to the amount of total PSA, and
a ratio of the amount of active free PSA to the amount of complexed PSA;
(iv) comparing the value of the ratio thus determined with a first predetermined differentiation value chosen corresponding to the type of ratio used, wherein the first differentiation value is representative of the values observed in patients suffering from a recognized benign pathology of the prostate;
(v) comparing the value of the ratio thus determined with a second predetermined differentiation value chosen corresponding to the type of ratio used, wherein the second differentiation value is representative of the values observed in individuals recognized to be suffering from an adenocarcinoma of the prostate; and
(vi) concluding from the results of the comparisons from steps (iv) and (v) whether there is present a benign pathology of the prostate or an adenocarcinoma of the prostate.
